(19) United States
(12) Patent Application Publication
(10) Pub. No.: US 2008/0058917 A1
(43) Pub. Date: Mar. 6, 2008

(54) Catheter for removing tissue from a hollow organ

(75) Inventors: Klaus Klingenbeck-Regn, Nurnberg (DE); Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/897,376

(22) Filed: Aug. 30, 2007

(30) Foreign Application Priority Data
Aug. 31, 2006 (DE) 10 2006 040 936

(51) Int. Cl. A61F 2/84

(52) U.S. Cl. 623/1.11

(57) ABSTRACT

The invention relates to a catheter for removing tissue from a hollow organ, comprising a lumen provided in the catheter shell for supplying to the nozzle-type opening a fluid used for removal of the tissue, via which the fluid is emitted into the area of tissue adjacent to the catheter tip via the opening provided, with an integrated image recording device being provided in the area of the catheter tip of which the recording area is directed onto the area of tissue adjacent to the outlet opening. Inventively a stent is provided in the area of the catheter tip, which can be expanded to set it via a balloon located underneath it inflatable via a further lumen.

### CROSS REFERENCE TO RELATED APPLICATIONS

This application claims priority of German application No. 10 2006 040 936.1 filed Aug. 31, 2006, which is incorporated by reference herein in its entirety.

### FIELD OF THE INVENTION

The invention relates to a catheter for removing tissue from a hollow organ, comprising a lumen provided in the catheter shell for supplying a fluid used for removal of the tissue to the nozzle-type outlet opening, via which the fluid is emitted into the outlet opening provided in the area of tissue adjacent to the catheter tip, with an integrated image recording device being provided in the area of the catheter tip the recording area of which is directed onto the area of tissue adjacent to the outlet opening.

### BACKGROUND OF THE INVENTION

Among the most frequent diseases with fatal consequences are vascular vessel diseases, especially the coronary infarction and the stroke. The coronary infarction is caused by diseases of the coronary vessels. In such cases arteriosclerotic plaque leads to a thrombocyte activation and local thrombus formation. This can lead to a total occlusion (blockage) of coronary vessels and thereby to a blocking of the blood flow. Similar mechanisms occur during a stroke and a closing-off of the peripheral vessels. The occlusion with a coronary infarction is currently treated in the majority of cases by a Percutaneous Transluminal Coronary Angioplasty (PCTA) in which the constrictions in coronary vessels are expanded with a balloon catheter. However this form of treatment frequently leads to restenosis, with the restenosis rate being able to be reduced by the use of stents which are placed in the widened-out constriction.

To further reduce the rate of restenosis there is the option of “drilling out” the previously narrowed vessel before the placement of the stent. An example of this is the DCA (Directional Coronary Atherectomy) method. The directional atherectomy device is a catheter system with a metal housing in which the actual cutting device, known as the “cutter”, is located. The “cutter”, comprising a conically ground knife, is connected via a flexible connection to an external motor. The knife rotates at a speed of 1500-2000 RPM. A balloon is mounted on one side of the metal housing, on the other side there is an opening. In the atherectomy the balloon is inflated which presses the knife and the opening into the plaque. The rotating knife can now be pushed forwards from outside against the tip of the atherectomy housing, which cuts out the plaque and pushes the plaque material into the tip of the atherectomy device. The balloon is then deflated, the atherectomy device rotated a little, so that the opening points in another direction of the plaque, after which the process is repeated. A disadvantage of this drilling facility, as with other drilling facilities, i.e. those for rotary ablation, lies in the ever-present danger of cutting through the vessel wall. This can be countered by a second catheter being introduced which contains an image recording device and via which the process of plaque removal can be monitored. However it is always necessary also to use a second catheter here. Even then a perforation of the vessel wall cannot be entirely excluded since a very direct, rapid process of tissue removal is still involved. There is also the problem with such drilling and milling devices of the material removed being hardly able or not able to be removed from the point of removal. This brings with it the risk of removed residues causing the formation of a new thrombosis, possibly even at other places in the vessel.

By contrast catheters are known which employ a fluid for removal of the tissue, preferably a salt solution. A lumen is provided in the catheter via which the removal fluid can be supplied via an external pump to the tip of the catheter, where a nozzle-type outlet opening is provided from which the fluid is emitted as a directed jet with adjustable pressure. This more or less sharp jet of fluid removes the tissue and flushes it away from the treatment point into the catheter for transport away, it is thus collected on the catheter side. Such a catheter device is for example known from DE 699 17 817 T2.

The catheter device described above is observed under x-ray control by means of contrast media with an angiography device during the treatment. The disadvantage of this monitoring method lies in the fact that in this case only the diameter of the vessel usable by the blood flow or the constriction respectively are shown in silhouette. If the vessel is completely closed-off the contrast media can no longer reach the area of the vessel after stenosis, this can no longer be displayed by the x-ray image. The medical personnel can thus not distinguish between plaque, thrombus and vessel wall during the intervention. An additional disadvantage is that the contrast media is also partly sucked out by the catheter device and thus x-ray imaging of the vessel is no longer possible. Monitoring in situ is possible by introducing an additional monitoring catheter with an imaging device, however the disadvantage of this is that a relatively expensive catheter must be introduced and handled in addition.

DE 691 32 093 T2 relates to atherectomy by means of a asymmetrical water jet catheter, in which, for monitoring options during of the procedure an ultrasound converter arrangement can be arranged at the remote end of the catheter. The catheter can be held on one side of the vessel by means of a balloon for removal of a deposit on the vessel wall. Furthermore an additional balloon can be provided, which is also attached to the catheter to provide an expansion of the vessel after the removal of the deposit.

### SUMMARY OF THE INVENTION

The object of the invention is to improve a catheter of the type mentioned above such that it allows removal of tissue in a hollow organ in a protective manner and its function is able to be observed easily during use, and also that it makes it possible for a vessel blockage to be completely treated by a one-off introduction of the inventive catheter.

To resolve this problem, the invention makes provision, with a catheter of the type mentioned at the start, for a stent to be provided in the area of the catheter tip, which can be expanded to place it via a balloon located underneath it which is able to be inflated via a further lumen. This metallic stent, made of stainless steel for example, can also be covered by a medical or therapeutic layer respectively (Drug Eluting Stent). Alternatively it can consist of a bioresorbable material which dissolves within a predetermined time. The Integration of such a stents thus enables a vessel blockage to be completely treated by a single insertion of the inventive catheter.

The outstanding feature of the inventive catheter is an imaging facility integrated into the tip, the recording area of which is directed towards the area of tissue treated with the emitted jet of fluid. This allows continuous observation both during the treatment and also beforehand and afterwards. The user also has the option on the one hand of using the integrated image recording device to orient themselves in situ, after obtaining the local circumstances directly via the image recording device. He can continuously check the removal procedure while undertaking it and can also immediately check the success of the treatment after it has been undertaken and make any necessary adjustments. An exact assignment of the stenosis to the position of the treatment area of the catheter is thus a simple matter.

A salt solution is preferably used as the removal fluid, other fluids can also be used or admixed, for example medicaments as well, e.g. heparin. The catheter itself, which is preferably equipped with an automatic catheter advance/withdrawal facility which moves the latter at a defined speed in the hollow organ or vessel respectively and also withdraws it again, is preferably embodied for transporting away the vessel particles removed by the jet of fluid through the catheter, preferably these are sucked out, for which purpose a separate suction lumen which is linked to a suction pipe is provided. The removed vessel particles are automatically flushed into this lumen.

According to a first alternate embodiment of the invention the imaging device can be an IVUS (Intravascular Ultrasound) device. In this case the images are recorded by ultrasound which means that on the tip side an ultrasound transmit and receive head is provided via which the images are recorded. The IVUS device can be designed as a rotating sensor or as a fixed sensor, with a plurality of the fixed sensors arranged in the shape of a ring of being able to be provided which can be controlled and interrogated cyclically with a multiplexer, with controlled timing for signal transmission. Sensors inclined forwards also enable the area in front of the catheter to be recorded.

Expediently with this embodiment a further lumen for supplying a contrast fluid to a further outlet opening provided in the area of the catheter tip is provided, via which the contrast fluid is emitted into the imaging area. Such a contrast medium, e.g. sulfur hexafluoride, forms temporary gas bubbles in the bloodstream and changes the reflection properties of the ultrasound signals, which leads to an improvement in image quality. Optionally a further lumen can be provided for supplying an x-ray contrast medium to enable an x-ray check to be undertaken in parallel if required.

An alternative to using an IVUS device can be to use an OCT (Optical Coherence Tomography) device as the image recording device. Here too a fixed or rotating OCT device can be provided with one or more sensors. As with the IVUS device, the OCT device is located in a correspondingly encapsulated area on the catheter, the ultrasound or light waves are coupled-in or coupled-out respectively via a corresponding ultrasound or light-transparent window.

A third alternate image recording device is an IVMRI (Intravascular Magnetic Resonance Imaging) device. With such a device an element generating a magnetic field is provided in the area of the catheter tip which undertakes a corresponding spin excitation with a similar effect to usual magnetic resonance tomography in the vessel, the corresponding response signals can be derived from the tissue via antennas and these are then processed into an image.

Regardless of which imaging device is integrated it is expedient to add one or more position sensors on the catheter tip side to allow the generation of three-dimensional images, be they IVUS, OCT or IVMRI images. The position sensors remedy the problem of no exact 3D representation of the vessel being obtained since the catheter is smaller than the vessel investigated and thus moves within the diameter of the vessel. This leads to movement artifacts in 3D image processing. With the aid of the position sensors the center line of the vessel and the vessel envelope can also be approximated. If the geometrical information of the center line is used and combined with the sensor position recorded during the imaging, artifacts in 3D imaging can be greatly reduced. The 3D co-ordinates of the center line and the sensor positions recorded during imaging are subtracted from each other. The result of the subtraction and is then used for each of the recorded 2D images for exact offset-corrected 3D reconstruction. The vessel envelope can be used for further image post processing steps. The mathematical envelope calculation can be undertaken as follows. The catheter inevitably collides with the boundary surfaces of the vessel during introduction and forward movement or retraction respectively. This creates a plurality of boundary points of which the coordinates are determined by the position sensors. The center line of the vessel is a one-dimensional line in a three-dimensional space. This can be computed on the basis of the recorded boundary points and described by a polynomial equation. In addition the minimum and maximum vessel diameter possible for the object investigated can be estimated.

The position sensors are preferably electromagnetic position sensors. The electromagnetic transmitter or alternately the electromagnetic receiver can be located in the catheter in this case. In addition the corresponding electromagnetic receivers or transmitters are accommodated outside the patient. A transmitter with an emission in the x-, y- and z direction to a receiver or conversely a receiver with a receive direction in the x, y and z direction is assigned to a transmitter, to allow position finding within the space. In specific cases the combination of two transmit devices to one receiver or vice-versa is also sufficient if the angular relationships are known and do not change.

To enable the catheter to be fixed in a particular position for treatment or image recording at least one balloon which can be inflated via a further lumen is expediently provided in the area of the catheter tip, preferably on the side opposite to the outlet opening for the fluid and where necessary the outlet opening for the contrast medium. The catheter uses this balloon to support it on the opposite organ or vessel wall respectively, whereas the treatment or imaging can be undertaken on the other side.

Furthermore one or more the elements interoperating with an external navigation magnetic field can be provided in the area of the catheter tip, especially one or more permanent magnets so as to provide the option of external magnetic field navigation which obviates the need for a guidance wire. The catheter can feature one or more electromagnets at the location of the permanent magnets. Where receive antennas are provided on the catheter side for receiving any image signals, these can be provided with a ferrite core so that with an appropriate dimensioning of these coils the unit can optionally be used as a receive antenna or as an electromagnet within the framework of external magnetic field navigation. A combination of the different options is also conceivable.

In a further development one guiding wire or a number of guiding wires to make possible an explicit bending of the catheter tip for an easier movement of the catheter tip through the vessel or such like can be provided, with the said guiding wires being routed to an external location and operated from there. In addition a definable tip bending in their direction can be achieved so that it is significantly easier to maneuver around any vessel bends etc.

Furthermore one or more x-ray-opaque markings can be provided on the catheter especially along the catheter shell, which make a precise detection of the catheter possible within the framework of a parallel x-ray monitoring. Furthermore one or more pressure and/or temperature sensors can be provided in the area of the catheter tip in order to monitor the temperature or the pressure respectively or a pressure gradient in the investigated and treated vessel or organ.

In order to record a possible movement of the patient and the artifacts arising from this during the examination, the option is basically provided, by using suitable sensors which are usually attached to the patient himself or herself, of recording the movement of the patient. Such a movement can be a rhythmic movement, for example the movement of the heart or the breathing movement, to which end corresponding recording means can be used via an ECG or a respiration strap. To remedy the breathing artifacts for example the breathing amplitude and frequency is determined via the breathing belt using appropriate sensors. Alternatively the amplitude and the frequency can be calculated from the envelope curve of the ECG signal. The image processing device then takes account of this information for artifact correction. Any systems can be used to record this type of patient movement, including optical systems for example, via which the position of a patient is scanned, magnetic or electromagnetic systems, e.g. using corresponding magnetic sensors on the patient or sensors in the form of RFID transponders etc.

There is also the option of manufacturing the catheter or the shell respectively etc. from materials which do not influence the image recording, i.e. of materials which for example shield against magnetic fields in the case of an IVMRI image recording device etc. The catheter can also be provided with a coating which reduces the frictional resistance during guidance. This coating can be made of silicon for example. Finally there is the option of decoupling the connections for the physiological sensors etc via a suitable electrical isolation from any mains voltage, in order not to endanger the patient. Optical decoupling is especially advantageous here. There is also the option of providing the catheter with a detection means, for example an RFID transponder via which the catheter can be exactly defined and which corresponds with an appropriate control device etc. for presetting the entire system in relation to the application of the catheter.

### BRIEF DESCRIPTION OF THE DRAWINGS

Further advantages, features and details of the invention are obtained from the exemplary embodiments described below as well as on the basis of the diagrams. The figures show:

FIG. 1 a basic diagram of an inventive catheter of a first embodiment,

FIG. 2 a basic diagram of an inventive catheter of a second embodiment and

FIG. 3 a basic diagram of the inventive catheter with assigned control and operating equipment.

### DETAILED DESCRIPTION OF THE INVENTION

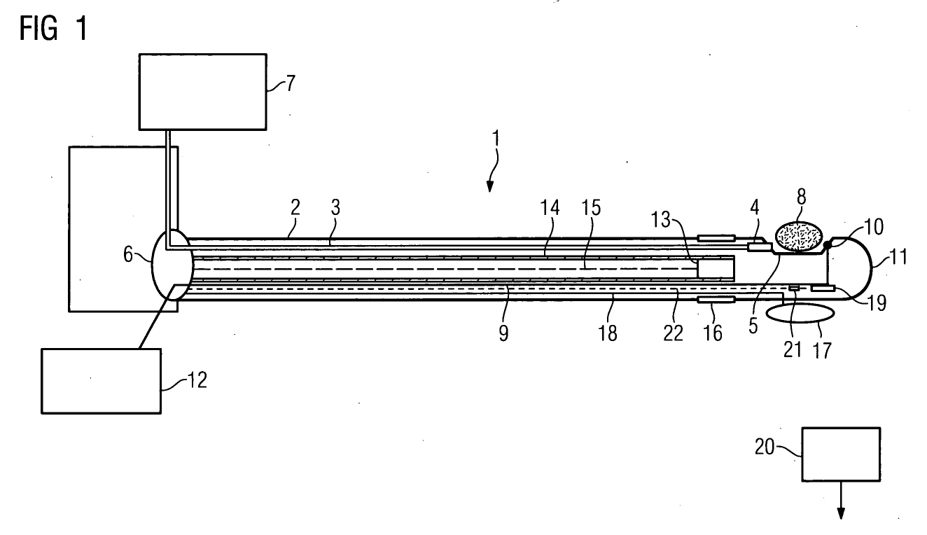

FIG. 1 shows a basic diagram of an inventive catheter 1, comprising a catheter shell 2, in which a first lumen 3 is provided, via which a fluid can be supplied to a nozzle-type outlet opening 4 which ends in an opening 5. From there the fluid, which is fed under pressure from a pump device 7 with fluid container which is connected by a releasable connection to the catheter at a corresponding coupling interface 6 is sprayed onto and external surrounding hollow organ on which in the example shown there is a thrombus 8. Via this the thrombus 8 or the tissue respectively is removed. Furthermore a second lumen 9 is provided, ending in an inlet opening 10 in the area of the catheter tip 11, and also releasably connected on the other side via the coupling interface 6 to a vacuum pump 12 along with storage container. Tissue removed is immediately sucked out again via this inlet opening 10 and the lumen 9, thus not remaining within the vessel.

Also provided in the example shown is an image recording device 13 which can optionally be an IVUS device, an OCT device or an IVMRI device. The signal line 15 to the image recording device 13 runs in a further lumen 14 which also ends in the coupling interface 6, from where it is connected to a suitable control and image processing device which will be discussed at greater length below.

The image recording device, regardless of its type, is arranged adjacent to a window 16 which, depending on the embodiment of the image recording device 13 is transparent for the issued ultrasound signals or alternating magnetic field and also for the reflected ultrasound signals or light signals or spin response signals emitted by the tissue to be picked up by the image recording device 13. The recording area of the image recording device 13 is directed in this case so that the opening 5 and the area of tissue adjacent to the outlet opening 4 which is treated with the fluid spray is completely recorded.

Also provided on the outside of the catheter shell 2 is a reversibly inflatable balloon 17 which is connected to a further lumen 18 via which it can be reversibly inflated or deflated. This lumen 18 too ends in the area of the coupling interface 6 and can be connected to a suitable pump or suction device which is not shown in any greater detail here. Via this reversibly inflatable balloon 17 the catheter tip of the naturally obviously significantly longer catheter 1 can be pressed against the opposing vessel wall and thereby fixed. This support automatically places the thrombus 8 in the area of the opening 5 enabling it to be easily treated with the fluid jet via the exit opening 4.

Finally a position sensor 19 is provided which can be detected using a position detection device 20. This position sensor provides the opportunity of determining the exact catheter tip position and thereby the spatial position of the thrombus or of the imaging and treatment section respectively of the catheter. Information can also be obtained in this manner for determining three-dimensional images of the treatment area.

Furthermore a pressure and/or temperature sensor 21 is provided in the example shown which communicates with a signal line 22 which ends in the coupling interface 6. Temperature or pressure information from the treatment area can be recorded via the sensor. This sensor is preferably located on the outer side of the catheter shell 2 to enable it to directly measure in the medium or on the tissue respectively.

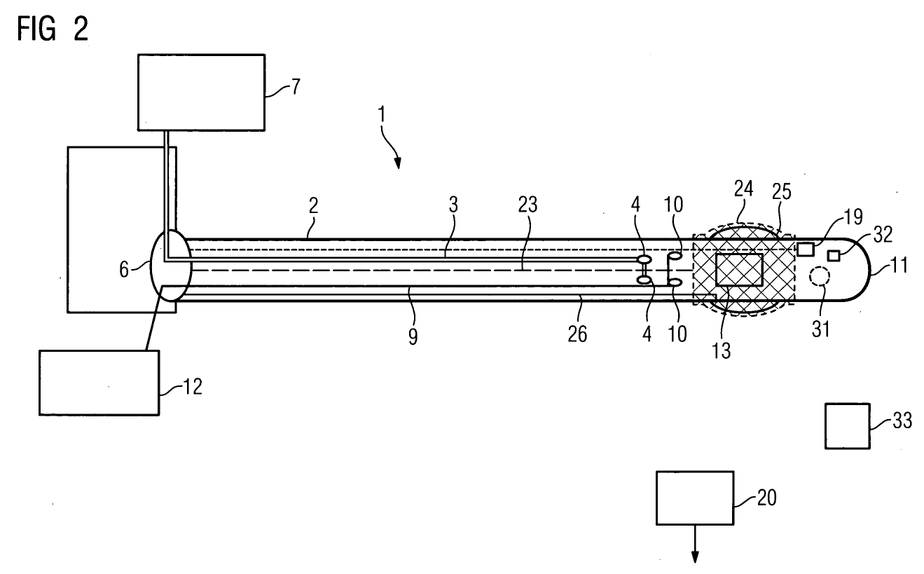

FIG. 2 shows a further embodiment variant of an inventive catheter 1 which to some extent corresponds to the catheter from FIG. 1 in its structure. Unlike the embodiment depicted in FIG. 1 no separate opening 5 is provided here however, instead of two nozzle-side outlet openings 4 are provided in the example shown on the side of the catheter shell from which the removal fluid is emitted under pressure. Assigned to these in the example shown are two inlet openings 10 via which the removed tissue is sucked away.

Here too an image recording device 13 is provided, which again can be any given imaging device. In the case of an IVUS or OCT or IVMRI device the device can either be fixed or can rotate. With a fixed arrangement a number of ultrasound or OCT sensors are arranged distributed around the circumference whereas with a rotating device a corresponding rotation drive is provided externally which can be connected via the coupling interface to a corresponding drive shaft 23 shown as a dashed line in this figure.

In each case the image recording area of the image recording device is also directed here towards the work area, i.e. the area of the outlet openings 4 and the inlet openings 10. Since these lie behind the image recording device 13 here the direction of view is the reverse of that shown for the embodiment depicted in FIG. 1.

Further shown in this embodiment is a stent 24 provided at the catheter tip 11 which can be expanded via a balloon 25 arranged below it which is connected via a further lumen 26 to an external pomp and extraction device. The balloon 25 is only shown in the collapsed state here by way of an example it runs radially around the catheter a shell 2 and can be inflated in the form of a ring to expand the stent 24.

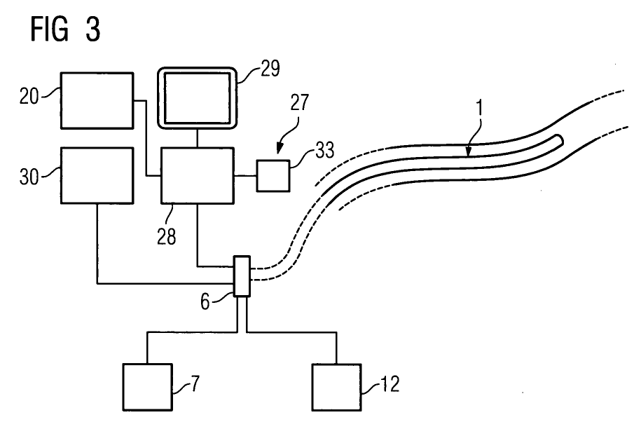

FIG. 3 finally shows a basic diagram of a complete catheter device 27 comprising an inventive catheter 1 which is connected by its coupling interface 6 to various items of external operating and imaging equipment. One device shown here is a control device 28 via which for example the operation of the imaging device 13 can be controlled, it also ensures the rotation of the rotating IVUS or OCT sensor as well as the corresponding issuing and receipt of signals. In it the corresponding received image signals are also edited and output on a monitor 29.

Also shown is the pump device 7 along with fluid container for supply of the removal fluid and also the vacuum pump 12 with the storage container for sucking out removed tissue or accumulations of thrombocytes.

Also shown it is an inflation and deflation device 30 via which for example the balloon 17 or alternatively or additionally also the balloon 25 can be inflated or deflated.

Also shown is the position detection device 20 which communicates with the control device 28.

The coupling of all elements via the coupling interface 6 is designed to ensure that any adverse effects on patients by electrical currents are avoided. Preferably optical signal couplers are used.

As is shown for example in FIG. 2 there is the option of providing a further image outlet opening 31 adjacent to the image recording facility 13 via which, via a further lumen routed in the catheter shell not shown in any greater detail here for example, a contrast fluid can be supplied to the image recording area. This is naturally only to be provided if the image recording device 13 used needs such a contrast fluid for enhanced image recording.

Also shown by way of example in FIG. 2 is an element 32 which interoperates with an external navigation device 33 for magnetic field navigation. This element 32 can for example be a permanent magnet.

This device 33, which is also only to be provided as an option, is also shown in FIG. 3; it communicates with the control device 28 or is controlled by this device respectively.

1.-11. (canceled)

12. A catheter for removing a tissue from a hollow organ of a patient, comprising:
a lumen arranged in a catheter shell through which a fluid used for removing the tissue is injected via an outlet opening arranged in a tip area of the catheter into an area of the tissue adjacent to the outlet opening;
an integrated image recording device arranged in the tip area of the catheter that records an image of the area of the tissue adjacent to the outlet opening; and
a stent arranged in the tip area of the catheter that is expanded and fixed via a balloon located below the stent.

13. The catheter as claimed in claim 12, wherein the image is continuously recorded during a process of the removal for monitoring and guiding the process.

14. The catheter as claimed in claim 12, wherein the image recording device is an IVUS device.

15. The catheter as claimed in claim 14, wherein a further lumen is provided in the catheter through which a contrast fluid is injected to an image recording area via a further outlet opening in the tip area of the catheter.

16. The catheter as claimed in claim 12, wherein the image recording device is an OCT device.

17. The catheter as claimed in claim 12, wherein the image recording device is an IVMRI device.

18. The catheter as claimed in claim 12, wherein a further balloon is provided in the tip area of the catheter for pressing and fixing the tip area of the catheter against an opposing vessel wall.

19. The catheter as claimed in claim 18, wherein the further balloon is arranged on a side opposite the outlet opening or the further outlet opening.

20. The catheter as claimed in claim 12, wherein a position sensor is provided in the tip area of the catheter.

21. The catheter as claimed in claim 12, wherein an element interoperating with an external navigation magnetic field is arranged in the tip area of the catheter.

22. The catheter as claimed in claim 12, further comprising a tensioning wire for explicitly bending the tip area of the catheter.

23. The catheter as claimed in claim 12, wherein an x-ray-opaque marking is arranged along the catheter shell.

24. The catheter as claimed in claim 12, wherein a pressure or a temperature sensor is arranged in the tip area of the catheter.

25. A catheter for removing a tissue from a hollow organ of a patient, comprising:
an integrated image recording device arranged in a tip area of the catheter that records an image of an area of the tissue for monitoring and guiding a process of the removal.

26. The catheter as claimed in claim 25, wherein the image is continuously recorded during the process of the removal.

27. The catheter as claimed in claim 25, further comprising a stent arranged in the tip area of the catheter that is expanded and fixed via a balloon located below the stent.

28. The catheter as claimed in claim 25, wherein the tissue is removed by a fluid injected via an outlet opening arranged in the tip area of the catheter into an area of the tissue adjacent to the outlet opening.

29. The catheter as claimed in claim 28, wherein the area of the tissue being recorded is the area of the tissue adjacent to the outlet opening.

30. A method for removing a tissue from a hollow organ of a patient using a catheter, comprising:
integrating an image recording device in a tip area of the catheter for recording an image of an area of the tissue; and
removing the tissue by the catheter with an aid of the recorded image.
